(19) United States
(12) Patent Application Publication
(10) Pub. No.: US 2020/0330021 A1
(43) Pub. Date: Oct. 22, 2020

(54) INCONTINENCE DETECTION SYSTEMS AND METHODS

(71) Applicant: KONINKLIJKE PHILIPS N.V., Eindhoven (NL)

(72) Inventor: Ali Akbar Ahmad SAMADANI, Eindhoven (NL)

(21) Appl. No.: 16/846,017

(22) Filed: Apr. 10, 2020

Related U.S. Application Data
(60) Provisional application No. 62/834,639, filed on Apr. 16, 2019.

(51) Int. Cl. A61B 5/20; A61B 5/00; A61B 5/053; G16H 10/60

(52) U.S. Cl. CPC A61B 5/202; A61B 5/746; A61B 5/053; A61B 2562/029; A61B 2562/0247; A61B 2562/0271; G16H 10/60

(57) ABSTRACT

A system is proposed for detecting incontinence of a subject. The system comprises an electronic textile sensing device arranged in an ambient environment of the subject comprising an incontinence detection sensor and a pressure detection sensor. The sensing device is configured to detect at least one incontinence-related parameter and at least one subject-specific parameter. The system comprises a processor connectively coupled to the electronic textile sensing device. The processor may process the at least one incontinence-related parameter and the at least one subject-specific parameter. The processor may identify the subject based on at least one of: the at least one incontinence-related parameter and the at least one subject-specific parameter, determine an incontinence event based on the at least one incontinence-related parameter, associate the incontinence event with the identified subject based on at least one of: the at least one incontinence-related parameter and the at least one subject-specific parameter, and generate an alert signal indicating the incontinence event and the identified subject. A method is proposed for detecting incontinence of a subject.

### CROSS-REFERENCE TO PRIOR APPLICATIONS

This application claims the benefit of U.S. Provisional Application No. 62/834,639, filed on 16 Apr. 2019. This application is hereby incorporated by reference herein.

### FIELD OF THE INVENTION

The present disclosure relates to incontinence detection systems and methods, and particularly, to incontinence detection systems and methods that use an electronic textile sensing device arranged in an ambient environment of a subject. More particularly, the present disclosure relates to incontinence detection systems and methods that associate incontinence event with an identified subject.

### BACKGROUND OF THE INVENTION

Incontinence refers to involuntary loss of urine or feces. Incontinence is common among varying populations, such as, for example, the aging population. However, prompt or immediate care is at times unavailable or unobtainable, such as when a person is unaware of the occurrence of an incontinent event or avoids seeking assistance due to a stigma surrounding incontinence. Incontinence, accompanied with lack of help with toileting and bathing, can lead to adverse psychosocial, medical, and health conditions, including pain, skin breakdowns, and infections. Incontinence can also adversely affect mental and psychological well-being.

Moreover, in a care facility with many patients, such as a hospital or a skilled nursing facility, it can be challenging to detect and/or monitor every patient for incontinence events. These challenges can be particularly important to patients with restricted mobility who may depend more intensely on caregivers to remediate incontinence events. Patients in private homes or smaller facilities may also face these challenges.

A wearable product or device may provide assistance with incontinence assistance. However, with individuals with chronic conditions, including those effecting cognitive wellbeing, monitoring based on wearables can be challenging, as adherence to wearables can be low. For example, an individual might not recognize or remember to wear a product or device.

Additionally, self-report of incontinence may not be reliable due to social stigma around incontinence.

### SUMMARY OF THE INVENTION

According to various embodiments, incontinence detection systems and methods may resolve the above mentioned problems by, for example, associating an incontinence event with an identified subject and provide the identified subject with suitable cares when incontinence event happens.

For this purpose, according to various embodiments, a system for detecting incontinence of a subject is proposed. The system may include an electronic textile sensing device arranged in an ambient environment of the subject comprising an incontinence detection sensor and a pressure detection sensor, wherein the sensing device may be configured to detect at least one incontinence-related parameter and at least one subject-specific parameter; and a processor connectively coupled to the electronic textile sensing device, wherein the processor may be configured to: identify the subject based on at least one of: the at least one incontinence-related parameter and the at least one subject-specific parameter; determine an incontinence event based on the at least one incontinence-related parameter; associate the incontinence event with the identified subject based on at least one of: the at least one incontinence-related parameter and the at least one subject-specific parameter; and generate an alert signal indicating the incontinence event and the identified subject.

In various embodiments, the incontinence detection sensor may include at least one of: a temperature sensor, a humidity sensor or a skin conductance sensor.

In various embodiments, the step of identifying the subject may include: storing a subject profile comprising subject-specific data of each incontinent individual; comparing the detected subject-specific parameter with corresponding subject-specific data; and identifying the subject when the detected subject-specific parameter is paired with one of the subject-specific data.

In various embodiments, the subject-specific data may include at least one of a pressure data or temperature data.

In various embodiments, the processor may be configured to determine the incontinence event by: comparing the detected incontinence-related parameter with a threshold; and determining the incontinence event when the detected incontinence-related parameter goes beyond the threshold.

In various embodiments, the processor may be configured to determine the incontinence event by analyzing a skin conductance parameter.

In various embodiments, the subject-specific data may include experimental pressure data collected from different incontinent individuals at different postures. In various embodiments, the subject-specific data may include pressure measurements mapped to actual physical build measurements using a regression model.

In various embodiments, the incontinence-related parameter may include at least one of a temperature parameter, a humidity parameter and a skin conductance parameter.

In various embodiments, the subject-specific parameter may include at least one of a temperature parameter and a pressure parameter.

In various embodiments, the processor may be further configured to estimate posture of a subject based on weight distribution detected by the pressure detection sensor.

In various embodiments, the processor may be further configured to track behavior of a subject based on reading from the electronic textile sensing device.

For this purpose, according to various embodiments, a method for detecting incontinence of a subject is proposed, the method may include: receiving at least one incontinence-related parameter and at least one subject-specific parameter; identifying the subject based on at least one of: the at least one incontinence-related parameter and the at least one subject-specific parameter; determining an incontinence event based on the at least one incontinence-related parameter; associating the incontinence event with the identified subject based on at least one of: the at least one incontinence-related parameter and the at least one subject-specific parameter; and generating an alert signal indicating the incontinence event and the identified subject.

In various embodiments, the at least one incontinence-related parameter and the at least one subject-specific parameter are detected by an electronic textile sensing device.

In various embodiments, the step of identifying the subject may include: storing a subject profile comprising subject-specific data of each incontinent individual; comparing the detected subject-specific parameter with corresponding subject-specific data; and identifying the subject when the detected subject-specific parameter is paired with one of the subject-specific data.

In various embodiments, the step of determining the incontinence event may include: comparing the detected incontinence-related parameter with a threshold; and determining the incontinence event when the detected incontinence-related parameter goes beyond the threshold.

In various embodiments, the method may include determining the incontinence event by analyzing a skin conductance parameter.

In various embodiments, the subject-specific data may include at least one of a pressure data or temperature data.

In various embodiments, the method may include estimating posture of a subject based on weight distribution detected by the pressure detection sensor.

In various embodiments, the method may include tracking behavior of a subject based on reading from the electronic textile sensing device.

### BRIEF DESCRIPTION OF THE DRAWINGS

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference numerals given below refer to the attached drawings, in which

FIG. 1 is a perspective view of an incontinence detection system located within a room of a care facility in accordance with an embodiment of the present invention;

FIG. 2 is an exemplary diagram of the incontinence detection system in accordance with an embodiment of the present invention;

FIG. 3 is a flowchart of a method for detecting incontinence of a subject in accordance with an embodiment of the invention; and

FIG. 4 is an embodiment of a computer readable medium storing instructions for the computer to perform the method of FIG. 3.

The figures are purely diagrammatic and not drawn to scale. In the Figures, elements which correspond to elements already described may have the same reference numerals.

### DETAILED DESCRIPTION OF EMBODIMENTS

The present disclosure includes systems and methods for electronic textile sensor based incontinence detection and monitoring that can alert caregivers, nurses, physicians, and/or the like to the occurrence of incontinence and the individual identification to assist in managing patients. The electronic textile sensor can be easily and effectively retrofitted into existing care facilities and/or an individual's own living environment and/or a private home. With the electronic textile sensor arranged in an ambient environment, the occurrence of the incontinence can be detected and monitored. Moreover, the individual's identification is further determined to provide a caregiver or the like essential information to remediate incontinence events when more than one party is present in a single room.

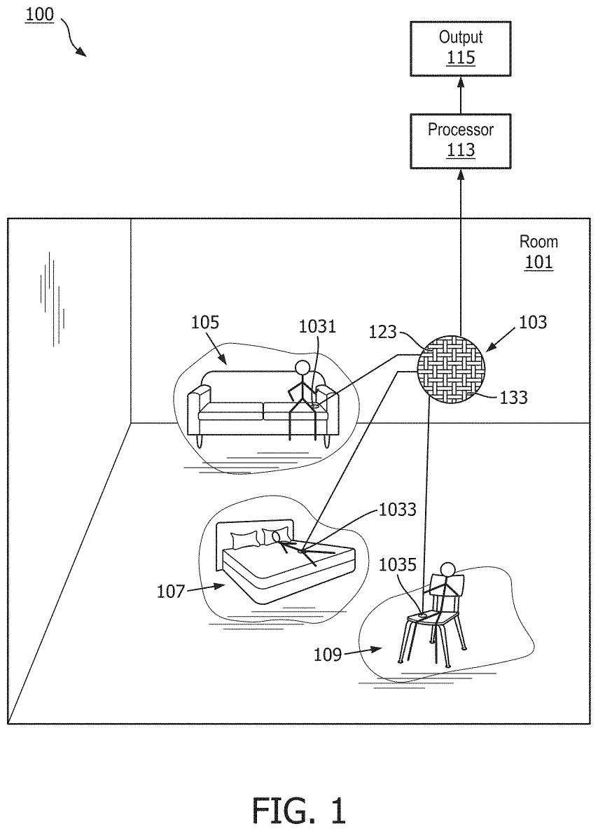

FIG. 1 shows an embodiment of an incontinence detection system 100 which may be at least partly located within a room 101 of a care facility, private home, or the like. An incontinence detection system 100 may not be located in a closed space such as the room of a care facility or the incontinent individual's own living environment or private home; it may also be located in an open space, including an outdoor space. That is to say, the incontinence detection system 100 may be arranged in a place where the incontinent individuals spend more time conducting activities.

The incontinence detection system 100 may comprise an electronic textile sensing device 103 arranged in an ambient environment of an individual or a subject for detecting at least one incontinence-related parameter, such as temperature, humidity, skin conductance, and/or the like, and at least one subject-specific parameter such as pressure, temperature, and/or the like.

The electronic textile sensing device 103 may include at least an incontinence detection sensor 123 and/or a pressure detection sensor 133 integrated in a textile substrate. The textile substrate may be made from many sources, such as for example, animal (e.g., wool), plant (e.g., cotton), or synthetic (e.g., nylon, polyester) that possess widely different structures and physical and chemical properties.

More specifically, as shown in FIG. 1, the incontinence detection sensor 123 and the pressure detection sensor 133 may include a plurality of sensors, respectively. In some embodiments, these sensors may be fabricated into the textile substrate. In some embodiments, these sensors may be in the form of a printed sensor integrated with the textile substrate. These sensors may be randomly or evenly distributed among the textile substrate.

As shown in FIG. 1, the electronic textile sensing device 103 may comprise multiple textile substrates 1031, 1033, 1035. Each of these textile substrates 1031, 1033, 1035 may be integrated with the incontinence detection sensor 123 and the pressure detection sensor 133. These textile substrates 1031, 1033, 1035 may be placed in the ambient environment of these incontinent individuals' room. As an example, these textile substrates 1031, 1033, 1035 are made in the form of film sheets with different contour shapes such as in the shape of round, rectangle and/or the like.

In various embodiments, these textile substrates 1031, 1033, 1035 may be placed on surface of at least a portion of a sofa 105, a bed 107 or a chair 109 where the incontinent individuals may sit/lie on, respectively. In various embodiments, these textile substrates 1031, 1033, 1035 may be fabricated as a portion of a sofa cover, a bed sheet, and/or a chair cover. When an individual conducts activities in the room, such as for example sitting or lying on a bed 107, sitting on a sofa 105 or sitting on a chair 109, particularly, sitting and/or lying on the surface of the electronic textile sensing device 103, the individual's at least one incontinence-related parameter and at least one subject-specific parameter may be detected.

It's advantageous that the above-mentioned ambient electronic textile sensors are less intrusive compared to the vision or camera sensors or wearables for detecting or monitoring incontinence events, and are generally more accepted by, for example, the elderly population. Furthermore, the flexibility of the electronic textile sensors allows them to conform to different surfaces allowing for efficient ambient incontinence detecting/monitoring. Additionally, these textile substrates are washable, removable and replaceable after being contaminated.

In an exemplary embodiment, the incontinence detection sensor 123 may include multiple temperature sensors, multiple humidity sensors and/or multiple skin conductance sensors.

The temperature sensors and/or the humidity sensors may be woven or embedded in the textile substrates 1031, 1033, 1035 and may be used to detect temperature and/or humidity parameters which can be used as the at least one subject-specific parameter.

When these textile substrates 1031, 1033, 1035 are in direct contact with an individual's skin, the skin conductance sensors may be used to detect a skin conductance parameter which can be used as the at least one subject-specific parameter.

More specifically, the skin conductance parameter may be indicative of electro-dermal activities of sweat glands representing sympathetic nervous system activities and may be measured through exosmotic measurement. This may in turn manifest psychological and physiological arousals. Electro-dermal activities may be used to estimate user's emotional state. Therefore, the skin conductance parameter may be used to assess an individual's emotional state including varying levels of affective arousal that can be indicative of agitation, discomfort, or sadness that might arise when the incontinent event occurs.

In various embodiments, the pressure detection sensor 133 may include multiple pressure sensors that may be used to detect pressure when the incontinent individual is sitting or lying on one or more textile substrates 1031, 1033 1035.

In various embodiments, several individuals may occupy a shared space, for example, room 101. Moreover, the individuals may conduct different types of activities in room 101. For example, one individual may sit on the chair 109, a second individual may lie on the bed 107 and/or a third individual may sit on the sofa 105. Accordingly, it may be necessary to identify the subject prior to and/or simultaneously while analyzing an incontinence event.

More specifically, when an individual is located on one of the textile substrates 1031, 1033, 1035 or when a pressure value is detected by the pressure detection sensor 133 of one of the textile substrates 1031, 1033, 1035, the electronic textile sensing device 103 of the corresponding textile substrate may be used to detect the at least one subject-specific parameter, such as a pressure parameter and/or a temperature parameter.

Under some circumstances, different individuals may have different body weight values and may cause different pressures amongst the pressure detection sensor(s) 133. In this case, the pressure parameter detected by the pressure detection sensor(s) 133 may be used as the at least one subject-specific parameter.

Under some circumstances, when the incontinent individuals live with, for example, a pet, in order to distinguish the incontinent individual from the pet, the temperature 123 and pressure sensor(s) 133 may further detect the temperature and pressure of the subject on the electronic textile sensing device 103. In this case, the temperature parameter and/or pressure parameter detected by the temperature sensor(s) 123 and/or pressure sensor(s) 133 may be used as the at least one subject-specific parameter.

When an individual occupies the surface of textile substrate(s) 1031, 1033, 1035 and when an incontinence event occurs, the incontinence detection sensor 123 may exposed to a moisture condition and the incontinence detection sensor 123 may be configured to detect the at least one incontinence-related parameter. More specifically, when incontinence event happens, a temperature and/or humidity in the proximity of the incontinence detection sensor 123 may increase suddenly and/or skin conductance detected by an incontinence detection sensor 123 may change suddenly.

For example, a temperature parameter, humidity parameter, and/or skin conductance parameter detected by the temperature, humidity, and/or skin conductance sensor(s) 123 may be used as the at least one incontinence-related parameter.

The incontinence detection system 100 may further include a processor 113. The processor 113 may be connectively coupled to the electronic textile sensing device 103. After receiving the above-mentioned at least one subject-specific parameter and at least one incontinence-related parameter. The processor 113 may be configured to process the at least one incontinence-related parameter and the at least one subject-specific parameter for determining an occurrence of an incontinence event as well as identifying the subject who is suffering from the incontinence event.

In various embodiments, the processor 113 may be in the form of a separate device, such as a PC, laptop, tablet, smartphone, workstation, server, and/or other computing device within a hospital network. In other embodiments, the processor 113 may include an output 115, such as a monitor, a display, a printer or a speaker, or may be combined with the output 115 into another device, in particular a hand-held device (e.g., a pad, a phone, a laptop and/or the like), a surveillance device and/or a vital signs monitoring device used or controlled by a caregiver, a nurse, a physician, and/or other third party. It is also possible that the processor 113 is part of a large network system that includes a plurality of computing devices, such as cloud or remote servers. In this case, the detected at least one incontinence-related parameter and the detected at least one subject-specific parameter may be transmitted wirelessly (e.g., Wi-Fi, Bluetooth, BLE, and/or cellular networks) to the processor 113 with a transmitter integrated into the textile substrates and a receiver arranged with the processor 113.

In some embodiments, the processor 113 may include a semiconductor-based microprocessor (in the form of a microchip) or a microprocessor, such as a digital signal processor (DSP), a Field Programmable Gate Array (FPGA), an Advanced RISC Machines (ARM) and/or the like. Under some circumstance, the processor 113 may be integrated into each of textile substrate 1031, 1033, 1035 together with an incontinence detection sensor 123 and/or pressure detection sensor 133. The incontinence detection sensor 123 and/or pressure detection sensor 133 may be connectively coupled with processor 113 in a wired manner or wirelessly (e.g., WLAN, Bluetooth, BLE, NFC, and/or the like). In this case, the detected at least one incontinence-related parameter and/or at least one subject-specific parameter may be transmitted to the processor 113 via the wired communication (e.g., Ethernet, cable, and/or the like) or wirelessly.

After receiving the at least one incontinence-related parameter and/or at least one subject-specific parameter, the processor 113 may identify a subject based on at least one of the at least one incontinence-related parameter and the at least one subject-specific parameter. The processor 113 may then determine an incontinence event based on the incontinence-related parameter. The processor 113 may then associate an incontinence event with an identified subject based on at least one of: the at least one incontinence-related parameter and the at least one subject-specific parameter. In addition, processor 113 may generate an alert signal indicating the incontinence event and the identified subject and transmit such an alert to a third party, such as, for example, a caregiver, a physician, a nurse, and/or the like.

More specifically, in various embodiments, the processor 113 may be configured to identify the subject by the following steps. First, the processor 113 may store a subject profile comprising subject-specific data of each individual in a group of individuals. The subject-specific data may include temperature data being obtained by measuring the temperature of the individual when the subject is in contact with the textile substrate 1031, 1033 or 1035, particularly, with the electronic textile sensing device 103.

The subject-specific data may include experimental pressure data stored in the processor 113. In some embodiments, the experimental pressure data may be collected in an experimental session from different individuals with different physical builds at different postures. The experimental pressure data may be specific to a particular individual and/or may be population-based such that a height, weight, and build of a particular individual may be used to determine a pressure map of that particular individual. A pressure map may be used to detect a specific individual and, upon identification, the pressure map may be individualized and updated for the specific individual using data obtained from each session associated with a specific individual where each session includes contact with textile substrate 1031, 1033 and/or 1035 incorporating at least one pressure sensor for a threshold amount of time.

In various embodiments, prior to deployment of the pressure detection sensor 133, the pressure detection sensor 133 may be calibrated. The calibration may include collecting experimental pressure data applied to the sensor proportional to varying levels of weights and areas. For example, each individual's experimental pressure data may be collected at different postures (e.g., sit, and different positions of sleep and laydown) for different physical builds (e.g., height, width, waistline, area, and lap size).

Pressure data also may be obtained by measuring objects resembling a diverse range of different physical builds. More specifically, prior to deployment of a pressure detection sensor 133, pressure detection sensor 133 may be calibrated. Calibration may include using a regression model to map the actual physical build measurements to their corresponding pressure measurements. These mapped pressure measurements through the regression model may construct the at least one subject-specific parameter.

In various embodiments, where new occupants might be added to a particular environment or space such as in the case of care facilities, a user profile may be created for the new occupant including information about their physical build (e.g., height, weight, bone structure, body measurements, and/or the like) and baseline body temperature (e.g., a range, and average, a median, and/or the like).

Since different individuals have different pressure data which may be used to distinguish the individuals from one another. The individuals may have different temperature data that may be used to distinguish one individual from another and/or an individual from another source of data such as a pet. In various embodiments, other types of data may also be used, such as subject-specific data, to distinguish one individuals from another or distinguish an individual from another source of data, such as a pet.

Second, the processor 113 may compare a reading or a detected subject-specific parameter from the at least one of the incontinence detection sensor 123 and the pressure detection sensor 133 with corresponding subject-specific data. For example, when a pressure parameter is used as the detected subject-specific parameter, the processor 113 may compare the pressure parameter with the pressure data of each individual respectively. Similarly, when a temperature parameter is used as the detected subject-specific parameter, the processor 113 may compare the temperature parameter with the temperature data of each individual respectively.

Third, the processor 113 may identify a subject when the detected subject-specific parameter is paired with one of the subject-specific data. For example, when the detected subject-specific parameter includes a pressure parameter, the pressure parameter may be compared against individual pressure data to determine whether the subject-specific parameter is the same as or substantially equal to one of the individuals' pressure data. For another example, when the detected subject-specific parameter comprises the temperature parameter, the temperature parameter may be compared against individual temperature data to determine whether the subject-specific parameter is the same as or substantially equal to one of the individuals' temperature data. The corresponding individual may then be identified as the subject and/or another subject and/or pet may be excluded.

In various embodiments, the detected temperature parameter and detected pressure parameter may refine the identification of the subject.

More specifically, in an exemplary embodiment, the processor 113 is configured to determine the incontinence event by comparing a detected incontinence-related parameter with a threshold. The threshold may include a temperature threshold and/or a humidity threshold detected by the temperature and/or humidity sensor 123 in a normal state when no incontinence event happens.

The processor 113 may determine an incontinence event when the detected incontinence-related parameter exceeds a predetermined threshold value. More specifically, when the detected temperature parameter and/or the humidity parameter exceeds a predetermined temperature threshold and/or the humidity threshold in the normal state.

In various embodiments, the processor 113 may determine an incontinence event by analyzing a skin conductance parameter. Moreover, the skin conductance parameter may reflect varying levels of psychological arousal and may be used to determine a subject's emotional state such as agitation, discomfort, or sadness. These emotional states might be a consequence of the occurrence of an incontinence event and the occurrence of the incontinence event is determined. Caregivers may be notified of the changes in skin conductance indicative of user's emotional state.

After the subject is determined to be a specific incontinent individual based on at least one of the incontinence-related parameter and the subject-specific parameter, the processor 113 may associate an incontinence event with an identified subject based on at least one of: at least one incontinence-related parameter and at least one subject-specific parameter.

The processor 113 may generate an alert signal indicating the incontinence event and the identified subject, which then may be transmitted (wirelessly or via a wired connection) to a third party, such as, for example, a caregiver, a nurse, a physician, and/or other third party. The alert signal may comprise a text signal, a voice signal, a visual signal, and/or any other type of output signal.

The alert signal may be sent to an output 115 of a caregiver's terminal as shown in FIG. 1. The output 115 may include, for example, a display, a printer, and/or a speaker. Based on the subject information, a caregiver may provide prompt care to the identified individual. For example, a caregiver may provide support to the identified individual with toileting and bathing and the caregiver may help remove the contaminated textile substrate or the contaminated sofa cover, bed sheet and/or chair cover and replace with a clean one.

In some embodiments, incontinence events for each subject may be stored and/or logged in the processor 113 and/or in a memory of a terminal or a server. A report on timings and frequency of the incontinence that are used by formal caregivers (e.g., primary care clinicians and physicians) may be generated for further examination. For example, when a subject is admitted to hospital, a report may be generated for a nurse and/or physician.

In this way, the automatic detection of incontinence event enabled by the above-mentioned incontinence detection system 100 addresses the social stigma around incontinence that causes an individual avoid seeking help and enables the delivery of prompt care when needed.

In some embodiments, the processor 113 may estimate posture of a subject based on weight distribution detected by the pressure detection sensor 133. The estimated posture may comprise, for example, a sitting and/or lying posture. For example, an individual may sit on an electronic textile sensing device 103 and/or may lie on the electronic textile sensing device 103 with supine or S-shape lying position. In some embodiments, the determination of the posture may be based on the area bearing weights that can be based on a determined number of active sensors at a time.

In some embodiments, the processor 113 may track behavior of a subject based on reading from the electronic textile sensing device. More specifically, in various embodiments, the sleeping and sitting behavior of each individual may be tracked based on a duration of continuous activation of a pressure detection sensor 133.

Tracking this behavior may provide additional detection and/or incontinence-related data. For example, an individual's behavior such as prolonged sleeping and/or sedentary behavior may be indicate pain, adverse health events, and/or depressive disorders. A prolonged sleeping posture may lead to development of pressure ulcer on the body areas in contact with bed. Using the pressure sensors 133, caregivers may be notified for timely changes of user's posture to avoid development of pressure ulcer. Furthermore, by continuous tracking of incontinence pattern that in turn can inform development of personalized care plan that is more user-aware and response-efficient. Such personalized care will help reduce pad dermatitis/rash and subsequent health problems.

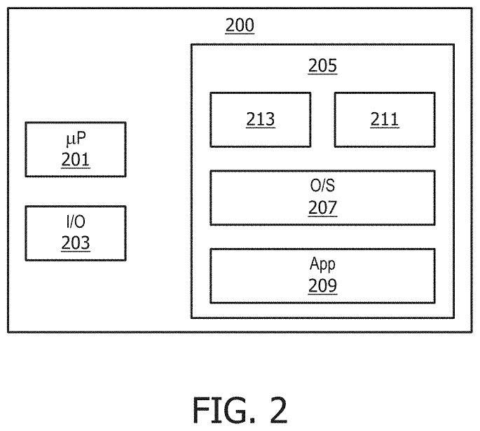

FIG. 2 illustrates a diagram of the incontinence detection system as described above. The incontinence detection system 200 may include, but is not limited to, PCs, workstations, laptops, smart phones, tables, PDAs, palm devices, servers, storages, and the like. Generally, in terms of hardware architecture, the incontinence detection system 200 may include one or more processors 201, memory 205, and one or more input/output (I/O) devices 203 that are communicatively coupled via an interface (not shown). The interface, such as a local interface may be, for example but not limited to, one or more buses or other wired or wireless connections, as is known in the art. The interface may have additional elements, such as controllers, buffers (caches), drivers, repeaters, and receivers, to enable communications. Further, the interface may include address, control, and/or data connections to enable appropriate communications among the aforementioned components.

The processor 201 may include a hardware device for executing software that can be stored in the memory 205. The processor 201 may include a custom made or commercially available processor, a central processor (CPU), a digital signal processor (DSP), and/or an auxiliary processor among several processors associated with the computer, and the processor 201 may be a semiconductor based microprocessor (in the form of a microchip) or a microprocessor. The processor 201 may be used to identify a subject based on at least one of: at least one incontinence-related parameter and at least one subject-specific parameter. The processor 201 may determine an incontinence event based on the at least one incontinence-related parameter. Processor 201 may associate the incontinence event with the identified subject based on at least one of: the at least one incontinence-related parameter and the at least one subject-specific parameter. Processor 201 may generate and transmit an alert signal indicating an incontinence event and an identified subject.

The memory 205 may include any one or combination of volatile memory elements (e.g., random access memory (RAM), such as dynamic random access memory (DRAM), static random access memory (SRAM), etc.) and/or non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 205 may incorporate electronic, magnetic, optical, and/or other types of storage media. Note that the memory 205 may have a distributed architecture, where various components are situated remote from one another, but can be accessed by the processor 201.

The software in the memory 205 may include one or more separate programs, each of which comprises an ordered listing of executable instructions for implementing logical functions. The software in the memory 205 includes a suitable operating system (O/S) 207, compiler 211, source code 213, and/or one or more applications 209 in accordance with various embodiments.

The application 209 may include numerous functional components such as computational units, logic, functional units, processes, operations, virtual entities, and/or modules. The application 209 may be an incontinence event monitoring application to communicate with the compiler 211, source code 213 and the suitable operating system 207 to control the processor 201 and I/O devices 203 to detect incontinence-related parameters and/or subject-specific parameters and output an alert signal when an incontinence event and associated subject is determined by the processor 201. The application 209 may comprise code means which is adapted, when said program is run on a computer, to perform the methods described herein.

The operating system 207 may control the execution of computer programs, and provides scheduling, input-output control, file and data management, memory management, and communication control and related services.

Application 209 may be a source program, executable program (object code), script, or any other entity comprising a set of instructions to be performed. When application 209 includes a source program, then the program may be translated via a compiler (such as compiler 211), assembler, interpreter, or the like, which may or may not be included within the memory 205, so as to operate properly in connection with the operating system 207. Furthermore, application 209 may be written as, for example, an object oriented programming language, which has classes of data and methods, or a procedure programming language, which has routines, subroutines, and/or functions, for example but not limited to, C, C++, C #, Pascal, BASIC, API calls, HTML, XHTML, XML, ASP scripts, JavaScript, FORTRAN, COBOL, Perl, Java, ADA, NET, and/or the like.

The I/O devices 203 may include input devices such as, for example, a mouse, keyboard, scanner, microphone, camera, and/or the like. In various embodiments, the I/O devices 203 may include the electronic textile sensing device 103 as described in FIG. 1. Furthermore, the I/O devices 203 may also include output devices, such as for example a printer, display, a microphone, a speaker, and/or the like. Finally, the I/O devices 203 may further include devices that communicate both inputs and outputs, for instance but not limited to, a network interface controller (NIC) or modulator/demodulator (for accessing remote devices, other files, devices, systems, or a network), a radio frequency (RF) or other transceiver, a telephonic interface, a bridge, a router, and/or the like. The I/O devices 203 may include components for communicating over various networks, such as the Internet and/or intranet.

When the computer is in operation, the processor 201 may execute software stored within the memory 205, to communicate data to and from the memory 205, and to generally control operations of the computer pursuant to the software. The application 209 and the operating system 207 may be read, in whole or in part, by the processor 201, buffered within the processor 201, and then executed.

When the application 209 is implemented in software, it should be noted that the application 209 may be stored on virtually any computer readable medium for use by or in connection with any computer related system or method.

Although the output is described above as a display, the output may be replaced and/or supplemented with sound, for example including speech generation to provide instructions, performance information or motivational encouragement, tactile (e.g. vibration) feedback, and/or other textual information. In an exemplary, the output is a display and a user interface may be designed to show the alert signal indicating the incontinence event and the identified subject as described in FIG. 1.

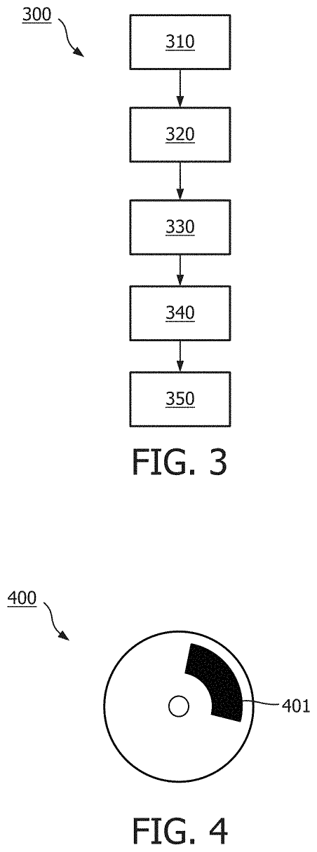

FIG. 3 shows a flowchart of a method for detecting incontinence of a subject in accordance with an embodiment of the invention. It is noted that the method 300 may, but does not need to, correspond to an operation of the incontinence detection system 100 or 200 as described with reference to FIGS. 1 and 2.

The method 300 includes receiving at least one incontinence-related parameter and/or at least one subject-specific parameter at block 310. The least one incontinence-related parameter and/or at least one subject-specific parameter may be detected by an electronic textile sensing device, such as sensing device 103.

The method 300 includes identifying a subject based one or more incontinence-related parameters and/or one or more subject-specific parameters at block 310. This may be accomplished by, for example, storing a subject profile comprising subject-specific data of each individual, comparing the detected subject-specific parameter with corresponding subject-specific data, and identifying the subject when the detected subject-specific parameter matches or is substantially similar to one of the subject-specific data. The subject-specific data may include, for example, at least one of a pressure data or temperature data.

The method 300 further includes determining an incontinence event based on a received at least one incontinence-related parameter at block 330. More specifically, the method may include comparing a detected incontinence-related parameter with a threshold, and determining an incontinence event when the detected incontinence-related parameter goes beyond the threshold. The method may comprise determining the incontinence event by analyzing a skin conductance parameter. The incontinence-related parameter may comprise the temperature parameter, the humidity parameter and/or the skin conductance parameter. The method 300 further includes associating the incontinence event with the identified subject in block 340. This association may be based on at least one of: an incontinence-related parameter and a subject-specific parameter.

The method 300 further includes generating an alert signal indicating the incontinence event and the identified subject at block 350. In various embodiments, the incontinence events for each subject may be stored/logged in a processor or in a memory of a server. A report on timings and frequency of the incontinence that are used by informal and/or formal caregivers (e.g., primary care clinicians and physicians) may be generated at any time, including, for example, when the subject is admitted to hospital.

The method 300 may further include estimating posture of a subject based on weight distribution detected by the pressure detection sensor.

The method 300 may further include tracking behavior of a subject based on reading from the electronic textile sensing device.

It will be appreciated that the above operation may be performed in any suitable order, e.g., consecutively, simultaneously, or a combination thereof, subject to, where applicable, a particular order being necessitated, e.g., by input/output relations.

The method 300 may be implemented on a computer as a computer implemented method, as dedicated hardware, or as a combination of both. As also illustrated in FIG. 4, instructions for the computer, e.g., executable code, may be stored on a computer readable medium 400, e.g., in the form of a series 401 of machine readable physical marks and/or as a series of elements having different electrical, e.g., magnetic, or optical properties or values. The executable code may be stored in a transitory or non-transitory manner. Examples of computer readable mediums may include memory devices, optical storage devices, integrated circuits, servers, online software, etc. FIG. 4 shows an optical disc 400.

It will be appreciated that the above description for clarity has described embodiments of the invention with reference to different functional units and processors. However, it will be apparent that any suitable distribution of functionality between different functional units or processors may be used without deviating from the invention. For example, functionality illustrated to be performed by separate units, processors or controllers may be performed by the same processor or controllers. Hence, references to specific functional units are only to be seen as references to suitable means for providing the described functionality rather than indicative of a strict logical or physical structure or organization. The invention can be implemented in any suitable form including hardware, software, firmware or any combination of these.

It is noted, that in this document the word ‘comprising’ does not exclude the presence of other elements or steps than those listed and the word ‘a’ or ‘an’ preceding an element does not exclude the presence of a plurality of such elements, that any reference signs do not limit the scope of the claims, that the invention may be implemented by means of both hardware and software, and that several ‘means’ or ‘units’ may be represented by the same item of hardware or software, and a processor may fulfill the function of one or more units, possibly in cooperation with hardware elements. Further, the invention is not limited to the embodiments, and the invention lies in each and every novel feature or combination of features described above or recited in mutually different dependent claims.

1. A system for detecting incontinence of a subject, comprising:
an electronic textile sensing device arranged in an ambient environment of the subject comprising an incontinence detection sensor and a pressure detection sensor, wherein the sensing device is configured to detect at least one incontinence-related parameter and at least one subject-specific parameter; and
a processor connectively coupled to the electronic textile sensing device, wherein the processor is configured to process the at least one incontinence-related parameter and the at least one subject-specific parameter, comprising:
identifying the subject based on at least one of: the at least one incontinence-related parameter and the at least one subject-specific parameter;
determining an incontinence event based on the at least one incontinence-related parameter;
associating the incontinence event with the identified subject based on at least one of: the at least one incontinence-related parameter and the at least one subject-specific parameter; and
generating an alert signal indicating the incontinence event and the identified subject.

2. The system of claim 1, wherein the incontinence detection sensor comprises at least one of: a temperature sensor, a humidity sensor or a skin conductance sensor.

3. The system of claim 1, wherein the processor is configured to identify the subject by:
storing a subject profile comprising subject-specific data of each incontinent individual;
comparing the detected subject-specific parameter with corresponding subject-specific data; and
identifying the subject when the detected subject-specific parameter is paired with one of the subject-specific data.

4. The system of claim 3, wherein the subject-specific data comprises at least one of a pressure data or temperature data.

5. The system of claim 1, wherein the processor is configured to determine the incontinence event by:
comparing the detected incontinence-related parameter with a threshold; and
determining the incontinence event when the detected incontinence-related parameter goes beyond the threshold.

6. The system of claim 1, wherein the processor is configured to determine the incontinence event by analyzing a skin conductance parameter.

7. The system of claim 1, wherein the subject-specific data comprises experimental pressure data collected from different individuals at different postures.

8. The system of claim 1, wherein the subject-specific data comprises pressure measurements mapped to actual physical build measurements using a regression model.

9. The system of claim 1, wherein the incontinence-related parameter comprises at least one of a temperature parameter, a humidity parameter and a skin conductance parameter.

10. The system of claim 1, wherein the subject-specific parameter comprises at least one of a temperature parameter and a pressure parameter.

11. The system of claim 1, wherein the processor is further configured to estimate posture of a subject based on weight distribution detected by the pressure detection sensor.

12. The system of claim 1, wherein the processor is further configured to track behavior of a subject based on reading from the electronic textile sensing device.

13. A method for detecting incontinence of a subject, comprising:
receiving, at a processor, at least one incontinence-related parameter and at least one subject-specific parameter from an electronic textile sensing device;
identifying, via the processor, the subject based on at least one of: the at least one incontinence-related parameter and the at least one subject-specific parameter;
determining, via the processor, an incontinence event based on the at least one incontinence-related parameter;
associating, via the processor, the incontinence event with the identified subject based on at least one of: the at least one incontinence-related parameter and the at least one subject-specific parameter;
generating, via the processor an alert signal indicating the incontinence event and the identified subject.

14. The method of claim 13, wherein the at least one incontinence-related parameter and the at least one subject-specific parameter are detected by an electronic textile sensing device comprising an incontinence detection sensor and a pressure detection sensor.

15. The method of claim 13, wherein the step of identifying the subject comprises storing a subject profile comprising subject-specific data of each incontinent individual;
comparing the detected subject-specific parameter with corresponding subject-specific data; and
identifying the subject when the detected subject-specific parameter is paired with one of the subject-specific data.

16. The method of claim 15, wherein the subject-specific data comprises at least one of a pressure data or temperature data.

17. The method of claim 13, wherein the step of determining the incontinence event comprises:
comparing the detected incontinence-related parameter with a threshold; and
determining the incontinence event when the detected incontinence-related parameter goes beyond the threshold.

18. The method of claim 13, wherein the method comprises determining the incontinence event by analyzing a skin conductance parameter.

19. The method of claim 13, wherein the method comprises estimating posture of a subject based on weight distribution detected by a pressure detection sensor.

20. The method of claim 13, wherein the method comprises tracking behavior of a subject based on reading from the electronic textile sensing device.
